(19) United States
(12) Patent Application Publication
(10) Pub. No.: US 2015/0276680 A1
(43) Pub. Date: Oct. 1, 2015

(54) METHOD FOR ASSESSING BIOAFFINITY OF ORGANIC BIOCARRIER FOR WASTEWATER TREATMENT

(71) Applicant: Nanjing University, Nanjing (CN)

(72) Inventors: Hongqiang REN, Nanjing (CN); Hui HUANG, Nanjing (CN); Lili DING, Nanjing (CN); Jinju GENG, Nanjing (CN); Ke XU, Nanjing (CN); Yan ZHANG, Nanjing (CN)

(21) Appl. No.: 14/672,262

(22) Filed: Mar. 30, 2015

(30) Foreign Application Priority Data
Mar. 31, 2014 (CN) 201410127046.X

(51) Int. Cl. G01N 29/02; G01N 33/543

(52) U.S. Cl. CPC G01N 29/022; G01N 33/54373

(57) ABSTRACT

A method for assessing bioaffinity of an organic biocarrier for wastewater treatment, the method including: 1) coating a matrix of an organic biocarrier on a standard chip to prepare a coated matrix chip; 2) preparing a testing water sample; 3) placing the coated matrix chip prepared in 1) in a quartz crystal microbalance (QCM), introducing the testing water sample prepared in 2) to the QCM, and measuring frequency data of the coated matrix chip under different overtones; and 4) fitting the frequency data of 3) using a Sauerbrey model to acquire variation of weights of an adlayer on a surface of the coated matrix chip with time; and comparing maximum fitted values of the weights of the adlayer on the surface of the coated matrix chip, to determine bioaffinity levels of the biocarriers to different testing water samples.

### CROSS-REFERENCE TO RELATED APPLICATIONS

Pursuant to 35 U.S.C. §119 and the Paris Convention Treaty, this application claims the benefit of Chinese Patent Application No. 201410127046.X filed Mar. 31, 2014, the contents of which are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, Mass. 02142.

### BACKGROUND OF THE INVENTION

1. Field of the Invention

The invention relates to the field of water treatment technology, and more particularly to a method for assessing bioaffinity of an organic biocarrier for wastewater treatment.

2. Description of the Related Art

A typical method for assessing bioaffinity of a biocarrier includes: placing the biocarrier in a sewage treatment system containing a wastewater to be treated (actual or analog wastewater), starting the biofilm formation using a closed circulation process or fast sludge discharge process, measuring removal rates of predominant pollutants (such as COD, NH₃—N, or particular pollutant factors), and determining whether the biofilm formation is successful based on observation results under a microscope. The assessment of the biocarrier's bioaffinity in the method is mainly based on the duration from the starting to the success of the biofilm formation, so it is time-consuming and inaccurate.

### SUMMARY OF THE INVENTION

In view of the above-described problems, it is one objective of the invention to provide a method for assessing bioaffinity of an organic biocarrier for wastewater treatment. The method utilizes a weight of an adlayer formed by soluble pollutants of the sewage on a surface of the coated matrix as parameters for assessing the bioaffinity of the biocarrier, thereby being easy to quantification and having good stability. Meanwhile, a quartz crystal microbalance (QCM) that is sensitive to interface changes is utilized to monitor the micro-precipitation of the soluble pollutants on the surface of the coated matrix, so that the method is timesaving and effective.

To achieve the above objective, in accordance with one embodiment of the invention, there is provided a method for assessing bioaffinity of an organic biocarrier for wastewater treatment. The method comprises:
1) coating a matrix of an organic biocarrier on a standard chip whereby preparing a coated matrix chip;
2) preparing a testing water sample;
3) placing the coated matrix chip prepared in 1) in a QCM, introducing the testing water sample prepared in 2) to the QCM, and measuring frequency data of the coated matrix chip under different overtones; and
4) fitting the frequency data of 3) using a Sauerbrey model to acquire variation of weights of an adlayer on a surface of the coated matrix chip with time; and comparing maximum fitted values of the weights of the adlayer on the surface of the coated matrix chip, whereby determining bioaffinity levels of the biocarriers to different testing water samples.

In a class of this embodiment, the coating method of 1) is spin-coating, vacuum coating, or monolayer self-assembly coating. In order to acquire good frequency responses, a thickness of a coated matrix is between 10 and 100 nm.

In a class of this embodiment, preparation of the testing water sample of 2) comprises: a) collecting a micro-polluted water, a municipal sewage, or an industrial wastewater as a water sample; and b) pre-extracting, centrifuging, and filtering the water sample; or directly centrifuging and filtering the water sample; or directly centrifuging the water sample; or directly filtering the water sample, whereby obtaining the testing water sample.

In a class of this embodiment, pre-extracting operation adopts resin extraction, heat extraction, sodium hydroxide extraction, or formalin fixation.

In a class of this embodiment, the testing water sample is degassed for between 5 and 15 min using an ultrasonic wave having a frequency of between 5 and 50 kHz before being introduced to the QCM.

In a class of this embodiment, when using the QCM for measurement in 3), testing conditions of the QCM are set as follows: a) a working temperature of the QCM is between 15 and 35° C. b) At least a fundamental frequency and a 3th overtone frequency are selected, and other overtone frequencies at least one selected from 5, 7, 9, 11, and 13 overtone frequencies. c) Solutions flowing through the coated matrix chip are in the following order: a background solution, the testing water sample, and the background solution. The background solution is a distilled water or a pure water. A flow rate of the solutions is set at between 50 and 300 μL/min in order to enhance an interface mass transfer and reduce the use of the solution.

Advantages according to embodiments of the invention are summarized as follows:

The method creatively utilizes the weight of the adlayer formed by the soluble pollutants of the sewage on the surface of the coated matrix as the parameter for assessing the bioaffinity of the biocarrier, thereby being prone to quantification and having good stability. Compared with the conventional assessing method, the method of the invention adopts the interface reaction, presuming that the sample is supplied at a minimum flow rate of 50 μL/min, it only consumes 1 mL of the testing water sample for 20 min of sample feeding, and thus, the consumption of the water sample is very small.

QCM that is sensitive to interface changes is utilized to monitor the micro-precipitation of the soluble pollutants on the surface of the coated matrix. The testing time generally does not exceed 1 hr, so that the method saves the testing time greatly and has broad application prospect in development of new recipes and performance assessment of the bio-organic biocarrier for wastewater treatment.

### BRIEF DESCRIPTION OF THE DRAWINGS

The invention is described hereinbelow with reference to the accompanying drawings, in which:

FIG. 1 is a chart showing variation of weights of an adlayer formed by (metabolic) products of soluble microbes of a domestic sewage on surfaces of a PA chip and a PS chip with time according to one embodiment of the invention; and

FIG. 2 is a chart showing variation of weights of an adlayer formed by extracellular polymers or polymers of an industrial wastewater on surfaces of a PA chip and a PS chip with time according to one embodiment of the invention.

### DETAILED DESCRIPTION OF THE EMBODIMENTS

For further illustrating the invention, experiments detailing a method for assessing bioaffinity of an organic biocarrier for wastewater treatment are described below. It should be noted that the following examples are intended to describe and not to limit the invention.

### Example 1

A method for assessing bioaffinity of an organic biocarrier for wastewater treatment is provided. In view that the conventional method for assessing the bioaffinity of the biocarrier consumes relatively much time, the method of the invention takes into consideration that as the original adhesion between the soluble pollutants of the wastewater and the surface of the biocarrier determines the subsequent formation and development of the biofilm, the purpose for assessing the bioaffinity of the biocarrier can also be realized by monitoring the difference in the adlayers formed by the soluble pollutants of the wastewater on the surfaces of different coated matrixes. The method of the invention creatively utilizes the weight of the adlayer formed by the soluble pollutants of the wastewater on the surface of the coated matrix as parameters for assessing the bioaffinity of the biocarrier, thereby being easy quantification and having good stability. The larger the weight of the adlayer is, the higher the bioaffinity of the biocarrier to the sewage or the wastewater is. Meanwhile, the sensitive variation of the interface of the QCM is adopted to dynamically monitor the characteristics of the micro-precipitation of the substances on the chip surface, thereby greatly saving the testing time and largely decreasing the amount of the testing water sample.

A method for assessing biocompatibilities of a polyamide (PA) biocarrier and a polystyrene (PS) biocarrier to a certain domestic sewage is performed as follows:

Step 1, preparation of a PA chip and a PS chip: the PA chip (a custom chip with a type of QSX 999) is purchased from Biolin Scientific, Sweden, and a coating method thereof is vacuum coating. The PS chip is a film chip formed by spin-coating a surface of a standard chip (a polished gold electrode with a diameter of 14 mm, purchased from Biolin Scientific, Sweden). Primary steps for preparing the PS chip are as follows: 1) a polystyrene solid is dissolved by tetrahydrofuran to yield a polystyrene solution having a concentration of 300 mg/L. 2) The standard chip is placed on a worktable of a spin-coating device (purchased from Chemat technology INC., USA, type KW-4A), and 300 μL of the prepared polystyrene solution is evenly dropped from above a center of the chip. 3) The spin-coating device is controlled to rotate at a rotational speed of 800 rpm for 10 s and then at the rotational speed of 3000 rpm for 50 s. 4) The PS chip is taken out after spin-coating the film and is then placed on a cleaning frame for natural drying.

A spectroscopic ellipsometry (M-2000V-ESM, J.A. Woollam Co., Inc.) is adopted to measure thicknesses of matrix films coated on the surfaces of the matrix chips, and incident angles are selected to be 70° and 80°. Thicknesses of films on the surfaces of the PA chip and the PS chip are 26.18±1.381 nm (MSE=2.525) and 30.65±1.836 nm (MSE=2.530), respectively, which satisfy requirements on the thicknesses of the matrix films.

Step 2, preparation of the testing water sample: the domestic sewage of this example is collected from a 150 m³/d domestic sewage treatment plant, centrifuged (6000 g, 10 min), and filtered using a 0.45 μm filter membrane, so that a testing water sampling containing soluble microbes (metabolic) products are obtained. Basic parameters are set as follows: a pH is 6.50, a temperature is 24.5° C., a conductivity is 652 μs/cm, and a soluble chemical oxygen demand (COD) is 310.2 mg/L, and the testing water sample is degassed for 10 min using an ultrasonic frequency of 20 kHz before being introduced to the QCM.

Step 3, the PS chip and the PA chip of step 1 is placed in the QCM, and the testing water sample prepared in step 2 is introduced. Herein the QCM is Q-Sense E1 QCM sensor provided by Biolin Scintific, Sweden and is capable of simultaneously monitoring a frequency variation and an energy dissipation factor and acquiring fitted values of weights of the adlayers by software. And specific testing steps are as follows:

1) Whether tubes of a flow module and a peristaltic pump of the QCM work normally and whether joints and interfaces are tightly connected are examined.

2) The PA or PS chip is correctly installed in the QCM.

3) Software QSoft401 (supporting software for the QCM) is opened, and the connection between the QCM and the computer is started. “Temperature” under the menu of “Acquisition” is clicked. “Manual” in the project of “Type of control” is chosen, and a working temperature of 25.0° C. of the QCM is input, and the temperature control is then activated.

4) “Setup Measurement” under the menu of “Acquisition” is clicked to open a dialog box. The PA and PS chips contained in the testing can be selected in the window of “Included crystals” (El system is “1”). Numbers representing Overtones of the chip required to be recorded are selected in “Included resonances”. “Find all resonances” is clicked, and “1st” (fundamental frequency), “3rd”, “5th”, and “9th” overtone frequencies are chosen. After that, “start measurement” under the menu of “Acquisition” is clicked.

5) A sample inlet tube of the QCM is placed in the air. The peristaltic pump is started, and a flow rate is set to be 150 μL/min. A sample outlet tube is disposed in a flask for collecting a waste solution in the experiment and submerged into the solution. When bubbles come out of the sample outlet tube, the peristaltic pump is temporarily stopped. Thereafter, the sample inlet tube is placed in a distilled water functioning as a background solution, the flow rate is set to be 150 μL/min, and operation of the peristaltic pump is resumed. Variation of the frequency (F) displayed on the software interface of QSoft401 is observed, and when F tends to change slightly, the peristaltic pump is temporarily stopped. The sample inlet tube is then displaced in the testing water sample prepared in step 2, and the operation of the peristaltic pump is resumed. When F tends to be stable again, the peristaltic pump is temporarily stopped. After that, the sample inlet tube is placed into the background solution, the operation of the peristaltic pump is resumed; and when F tends to be stable, the peristaltic pump is temporarily stopped. A total testing time of this step is less than 25 min.

6) After the testing, inner walls of the tubes are washed by a large amount of a pure water and dried by aerating nitrogen. The chip is removed. A sealing ring is taken out of the sample cell, washed by an ultrasonic wave and dried, and the sample cell is dried by nitrogen aeration. The clean sealing ring is put back into the sample cell, and a flow cell and a sample table are installed.

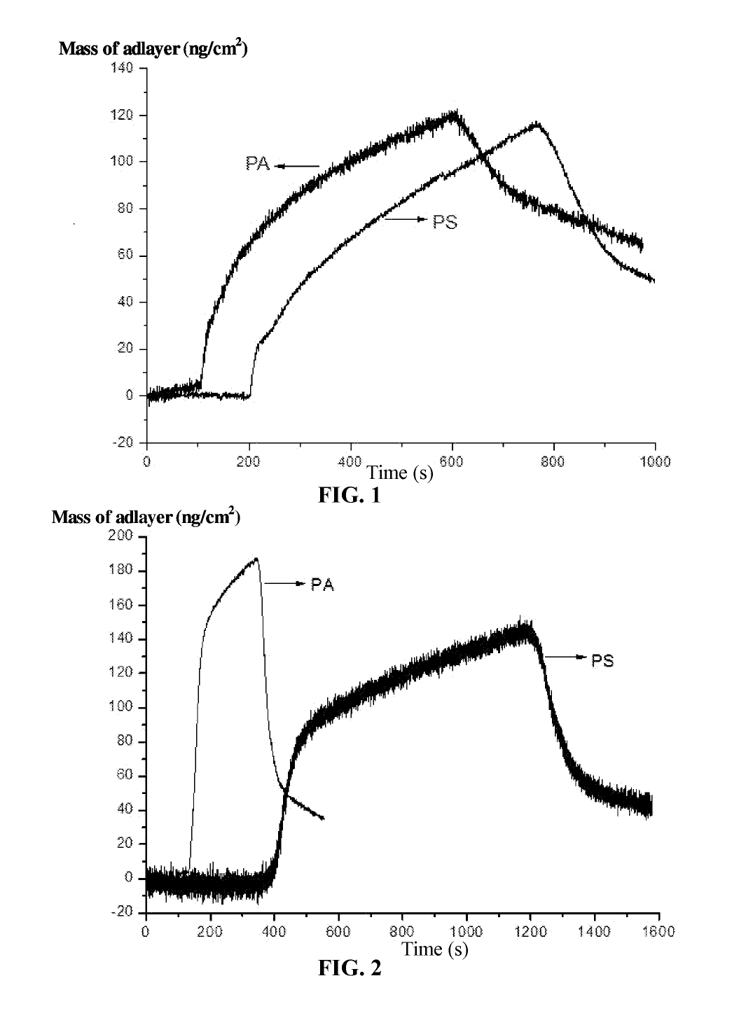

Step 4, the 3th overtone frequency data obtained from step 3 are adopted, Sauerbrey model is used for fitting, and the variation of the weight of the adlayer is obtained. Specific operation process is conducted as follows: an analysis tool of the software is open, “Sauerbrey” is chosen from “Data”, and “Areal mass” is chosen from the project of “Calculate”. “F_{—}1:3” is chosen from “Frequency column”, and corresponding output column “sau mass” is input into “Choose output”, and “Calculate” is clicked. Thus, the fitted data of the weight variation of the adlayer is acquired, and FIG. 1 is obtained by charting the fitted data in relation to the time. As shown in FIG. 1, maximum weights of the adlayer on the surfaces of the PA and PS chips are 123.0751 ng/cm² and 117.6106 ng/cm², respectively, thereby determining that the PA biocarrier has higher bioaffinity to the water sample of this example.

### Example 2

A method for assessing biocompatibilities of the PA biocarrier and the PS biocarrier to a certain industrial wastewater is conducted. The method of this example is basically the same as that of Example 1 and difference of the method is described as follows:

Step 1, preparation of a PA chip and a PS chip: the PA chip is purchased from Biolin Scientific, Sweden. The PS chip is a film chip formed by spin-coating the surface of the standard chip. Primary steps for preparing the PS chip are as follows: 1) a polystyrene solid is dissolved by tetrahydrofuran to yield a polystyrene solution having a concentration of 100 mg/L; 2) the standard chip is placed on the worktable of the spin-coating device, and 1000 μL of the prepared polystyrene solution is evenly dropped from above the center of the chip; 3) the spin-coating device is controlled to rotate at a rotational speed of 400 rpm for 15 s and then at the rotational speed of 1000 rpm for 60 s; and 4) the PS chip is taken out after spin-coating the film and is then placed on the cleaning frame for natural drying.

The spectroscopic ellipsometry is adopted to measure thicknesses of matrix films coated on the surfaces of the matrix chips, and incident angles are selected to be 70° and 80°. Thicknesses of films on the surfaces of the PA chip and the PS chip are 29.21±1.452 nm (MSE=2.364) and 32.64±2.315 nm (MSE=2.748), respectively, which satisfy requirements on the thickness of the matrix films.

Step 2, preparation of the testing water sample: the wastewater of this example is collected from a 6000 m³/d wastewater treatment plant in a chemical industrial park, performed with resin extraction (75 g of a cation exchange resin/g of a volatile solid), centrifuged (3000 g, 20 min), and filtered using a 0.22 μm filter membrane, so that a testing water sampling containing extracellular polymers or polymers are obtained. Basic parameters are set as follows: the pH is 7.18, the temperature is 28.2° C., the conductivity is 14.61 μs/cm, and the soluble COD is 789.4 mg/L, and the testing water sample is degassed for 15 min using an ultrasonic frequency of 5 kHz before being introduced to the QCM.

Step 3, the working temperature of the QCM is set to be 35° C. The 1st (fundamental frequency), 3rd, 5th, 7th, and 9th overtone frequencies are selected. The flow rate of the fluid is set to be 50 μL/min.

Step 4, the 3th overtone frequency data obtained from step 3 are adopted, Sauerbrey model is used for fitting, and the variation of the weight of the adlayer is obtained (as shown in FIG. 2). It is known from FIG. 2 that maximum weights of the adlayers formed by the extracellular polymers or polymers of the wastewater on the surfaces of the PA and PS chips are 187.9644 ng/cm² and 153.7187 ng/cm², respectively, thereby determining that the PA biocarrier has higher bioaffinity to the water sample of this example.

### Example 3

A method for assessing biocompatibilities of the PA biocarrier and the PS biocarrier to a certain industrial wastewater is conducted. The method of this example is basically the same as that of Example 1 and difference of the method is described as follows:

Step 1, preparation of a PA chip and a PS chip: the PA chip is purchased from Biolin Scientific, Sweden. The PS chip is a film chip formed by spin-coating the surface of the standard chip. Primary steps for preparing the PS chip are as follows: 1) a polystyrene solid is dissolved by tetrahydrofuran to yield a polystyrene solution having a concentration of 1000 mg/L; 2) the standard chip is placed on the worktable of the spin-coating device, and 50 μL of the prepared polystyrene solution is evenly dropped from above the center of the chip; 3) the spin-coating device is controlled to rotate at a rotational speed of 1000 rpm for 3 s and then at the rotational speed of 1500 rpm for 30 s; and 4) the PS chip is taken out after spin-coating the film and is then placed on the cleaning frame for natural drying.

The spectroscopic ellipsometry is adopted to measure thicknesses of matrix films coated on the surfaces of the matrix chips, and incident angles are selected to be 70° and 80°. Thicknesses of films on the surfaces of the PA chip and the PS chip are 24.35±1.328 nm (MSE=2.034) and 26.334±2.082 nm (MSE=2.436), respectively, which satisfy requirements on the thickness of the matrix films.

Step 2, preparation of the testing water sample: the wastewater of this example is collected from a 6000 m³/d wastewater treatment plant in a chemical industrial park, performed with heat extraction (100° C., 10 min), centrifuged (8000 g, 5 min), and filtered using a 0.45 pm filter membrane, so that a testing water sampling containing extracellular polymers or polymers are obtained. The testing water sample is degassed for 5 min using an ultrasonic frequency of 50 kHz before being introduced to the QCM.

Step 3, the working temperature of the QCM is set to be 15° C. The 1st (fundamental frequency), 3rd, 5th, 7th, and 13th overtone frequencies are selected. The pure water is used as the background solution. The flow rate of the fluid is set to be 300 μL/min.

Step 4, the 3th overtone frequency data obtained from step 3 are adopted, Sauerbrey model is used for fitting, and the variation of the weight of the adlayer is obtained. The maximum weights of the adlayers formed by the extracellular polymers or polymers of the wastewater on the surfaces of the PA and PS chips are 147.8524 ng/cm² and 118.3204 ng/cm², respectively, thereby determining that the PA biocarrier has higher bioaffinity to the water sample of this example.

The method for assessing biocompatibilities of the biocarriers for sewage treatment, as described in above Examples 1-3, has short testing time, requires a small amount of the water sample, thereby being prone to quantification and having stability. Besides, the method has broad application prospect in new recipe development and performance assessment of the biocarrier for water treatment and is helpful for propelling the standardization for performance assessment of the biocarrier.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. A method for assessing bioaffinity of an organic biocarrier for wastewater treatment, the method comprising:
1) coating a matrix of an organic biocarrier on a standard chip whereby preparing a coated matrix chip;
2) preparing a testing water sample;
3) placing the coated matrix chip prepared in 1) in a quartz crystal microbalance (QCM), introducing the testing water sample prepared in 2) to the QCM, and measuring frequency data of the coated matrix chip under different overtones; and
4) fitting the frequency data of 3) using a Sauerbrey model to acquire variation of weights of an adlayer on a surface of the coated matrix chip with time; and comparing maximum fitted values of the weights of the adlayer on the surface of the coated matrix chip, whereby determining bioaffinity levels of the biocarriers to different testing water samples.

2. The method of claim 1, wherein in 1), a coating method is spin-coating, vacuum coating, or monolayer self-assembly coating; and a thickness of a coated matrix is between 10 and 100 nm.

3. The method of claim 2, wherein preparation of the testing water sample of 2) comprises: a) collecting a micro-polluted water, a municipal sewage, or an industrial wastewater as a water sample; and b) pre-extracting, centrifuging, and filtering the water sample; or directly centrifuging and filtering the water sample; or directly centrifuging the water sample; or directly filtering the water sample, whereby obtaining the testing water sample.

4. The method of claim 3, wherein the pre-extracting adopts resin extraction, heat extraction, sodium hydroxide extraction, or formalin fixation.

5. The method of claim 2, wherein the testing water sample is degassed for between 5 and 15 min using an ultrasonic wave having a frequency of between 5 and 50 kHz before being introduced to the QCM.

6. The method of claim 3, wherein the testing water sample is degassed for between 5 and 15 min using an ultrasonic wave having a frequency of between 5 and 50 kHz before being introduced to the QCM.

7. The method of claim 5, wherein when using the QCM for measurement in 3), testing conditions of the QCM are set as follows:
a) a working temperature of the QCM is between 15 and 35° C.;
b) at least a fundamental frequency and a 3th overtone frequency are selected, and other overtone frequencies at least one selected from 5, 7, 9, 11, and 13 overtone frequencies; and
c) solutions flowing through the coated matrix chip are in a following order: a background solution, the testing water sample, and the background solution; the background solution is distilled water or pure water; and a flow rate of the solutions is set at between 50 and 300 μL/min.

8. The method of claim 6, wherein when using the QCM for measurement in 3), testing conditions of the QCM are set as follows:
a) a working temperature of the QCM is between 15 and 35° C.;
b) at least a fundamental frequency and a 3th overtone frequency are selected, and other overtone frequencies at least one selected from 5, 7, 9, 11, and 13 overtone frequencies; and
c) solutions flowing through the coated matrix chip are in a following order: a background solution, the testing water sample, and the background solution; the background solution is distilled water or pure water; and a flow rate of the solutions is set at between 50 and 300 μL/min.
